(19) United States
(12) Patent Application Publication
(10) Pub. No.: US 2008/0124449 A1
(43) Pub. Date: May 29, 2008

(54) Food for Preventing Life Style-Related Diseases

(76) Inventors: Miho Ishii, Hyogo (JP); Masatoshi Shiojiri, Hyogo (JP); Satohiro Tanaka, Kyoto (JP); Tomoko Tanaka, Kyoto (JP); Xiaoli Liu, Hyogo (JP)

Correspondence Address: RENNER OTTO BOISSELLE & SKLAR, LLP, 1621 EUCLID AVENUE, NINETEENTH FLOOR, CLEVELAND, OH, 44115, US

(21) Appl. No.: 11/815,656

(22) PCT Filed: Dec. 2, 2005
(86) PCT No.: PCT/JP05/22610

§ 371 Date: Aug. 7, 2007

(30) Foreign Application Priority Data
Feb. 10, 2005 (JP) 2005-034342
Jul. 27, 2005 (JP) 2005-218039

(51) Int. Cl. A23J 1/14; C07K 2/00; C07K 5/08

(52) U.S. Cl. 426/656; 530/300; 530/331

(57) ABSTRACT

A food for preventing life style-related diseases of the present invention contains a mung bean protein degradation product. The mung bean protein degradation product is obtained by hydrolyzing a mung bean-derived protein with a protease. In the mung bean protein degradation product, the dipeptide or tripeptide content in the entire peptides is preferably 10% or more. The food for preventing life style-related diseases of the present invention is preferable for preventing in particular hypertension, arteriosclerosis, and obesity, for example. The present invention also provides an anti-hypertensive agent, an anti-arteriosclerotic agent, a vascular intimal hypertrophy inhibitor, and an anti-obesity agent containing the mung bean protein degradation product. Furthermore, the present invention provides a diet food containing the mung bean protein degradation product. The mung bean protein degradation product is obtained from naturally-occurring materials that have been conventionally used as foods, and thus it has little side effects and a high safety. Furthermore, this degradation product can be produced from a by-product obtained in the process of producing bean-starch vermicelli, and thus it is environmentally friendly in view of utilizing waste materials, and is expected to contribute to the recycling-based society.

### TECHNICAL FIELD

The present invention relates to a food for preventing life style-related diseases. More specifically, the present invention relates to an anti-hypertensive agent, an anti-arteriosclerotic agent, a vascular intimal hypertrophy inhibitor and an anti-obesity agent, in which a mung bean protein degradation product is contained, and a food, a drug and a quasi-drug, in which the anti-hypertensive agent, the anti-arteriosclerotic agent, the vascular intimal hypertrophy inhibitor or the anti-obesity agent is contained. The present invention also relates to a food having a diet effect, in which a mung bean protein degradation product is contained.

### BACKGROUND ART

Recently, life style-related diseases increase and have become a social problem. Life style-related diseases are required to be prevented/improved in daily life. For this purpose, it is desirable to ingest functional substances having a high degree of safety. Since foods contain various physiologically active substances, there is a growing interest in food-derived functional substances and functional foods.

Hypertension, which is one type of life style-related diseases, has few subjective symptoms. However, when left without any treatment, hypertension promotes arteriosclerosis, which serves as a factor to induce serious diseases such as myocardial infarction, cerebral infarction, and renal disease. Thus, it is important to improve and prevent hypertension. Recently, a relationship between the morning hypertension and the onset of cerebro- and cardiovascular diseases has been attracting attention, and the importance of blood pressure control in the early morning for suppressing the onset of cerebro- and cardiovascular diseases has been pointed out (S. Noguchi et al., BIO Clinica, 2003, Vol. 18, No. 5, pp. 63-67). Thus, there is an increasing need for a long-acting hypotensive agent that can suppress the blood pressure until morning when taken before sleep. Furthermore, in health control in daily life, not only a temporary hypotensive action, but also a chronic action by long-term administration and sustainability of the action after drug termination are desired.

Based on a relationship with the blood pressure increasing mechanism or the hypertension onset/development mechanism, it is considered that a substance having an angiotensin converting enzyme (ACE) inhibitory action or a reactive oxygen species removal action is expected to have an anti-hypertensive effect. Thus, as a first step of an evaluation of the anti-hypertensive effect, an ACE inhibitory potency or a reactive oxygen species removal ability is evaluated in vitro (Japanese Laid-Open Patent Publication Nos. 2003-24012 and 9-309833). However, a substance is variously affected in the living body, and thus an in vitro evaluation is merely an evaluation of actions of the substance from one aspect, and the evaluation results are not necessarily reflected in vivo. Accordingly, it is difficult to evaluate, only in vitro, various aspects in the body. Thus, it is desirable to perform an evaluation also in vivo.

Recently, food-derived substances are investigated as anti-hypertensive agents, and some substances are confirmed to have an action of lowering blood pressure or an action of suppressing blood pressure elevation in vivo. Examples of these substances include a product obtained by hydrolyzing wheat glutenin with chymotrypsin (Japanese Laid-Open Patent Publication No. 4-187643), and a sesame protein degradation product (Japanese Laid-Open Patent Publication No. 7-69922). Their hypotensive action is observed in a single administration to spontaneous hypertensive rats (SHRs), but the effect becomes less obvious within eight hours after the administration. A tripeptide derived from a rapeseed protein degradation product is also reported as a long-acting hypotensive agent, but its effect is lowered in a similar manner to the above (Japanese Laid-Open Patent Publication No. 2004-51636). In addition, a purified product of a soybean protein degradation product (Japanese Laid-Open Patent Publication No. 5-339166) and a caseinoglycopeptide contained in cheese whey and the like (Japanese Laid-Open Patent Publication No. 6-345664) have been confirmed to have an effect of suppressing blood pressure elevation in repeated administrations to SHRs, but sustainability of this effect has not been discussed.

As a substance that exhibits, after a long-term administration, sustainability of an effect of suppressing blood pressure elevation after drug termination, a lactobacillus-treated product derived from skim milk has been reported (Y. Nakamura et al., Biosci. Biotech. Biochem., 1996, Vol. 60, No. 3, pp. 488-489). When the lactobacillus-treated product was continuously administered together with a diet to SHRs for 16 weeks, suppression of the blood pressure was observed over 48 hours after termination of the administration. Furthermore, it has been reported that when a sardine-derived peptide was continuously administered to humans for four weeks, sustainability after drug termination in the hypotensive action was observed over three weeks after (Japanese Laid-Open Patent Publication No. 11-228599). However, the samples used in this experiment are obtained by degrading a sardine protein and then fractionating the resultant using an ODS resin, and thus it takes cost and effort for production.

On the other hand, arteriosclerosis, which is one type of life style-related diseases, is increasing, as the dietary lifestyle in Japan is shifting to the Western style, and as opportunities to ingest high-calorie foods are significantly increasing. Such high-calorie foods usually contain a large amount of fats and cholesterol, and thus excessive ingestion of these foods on a daily basis gives a harmful influence on the blood vessels and induces various diseases. It constitutes a factor to increase arteriosclerotic diseases such as ischemic heart disease, cerebrovascular disorder, and chronic obstructive arteriosclerosis, in combination with smoking, drinking, stress, aging, and the like. The onset of these diseases may jeopardize the lives of patients, or may interfere with daily life by troubles caused by the after-effects, deteriorating the quality of life (QOL). Furthermore, it is feared that diseases such as arteriosclerosis due to aging will increase in the future. In order not to cause onset of these diseases, from the viewpoint of preventive medicine, continuous ingestion of food materials derived from naturally-occurring product having an anti-arteriosclerotic action in daily life is effective. Prevention of arteriosclerosis and its accompanying diseases is one of the best means for suppressing deterioration of the QOL in aging and for reducing rapidly increasing insurance and medical costs.

Examples of foods that are being developed for the purpose of preventing arteriosclerosis include an extract from at least one of rice bran, Siraitia grosvenorij (rakanka), Lyophyllum aggregatum (shimeji mushroom), chrysanthemum, rye, white birch, and Alpinia speciosa (Japanese Laid-Open Patent Publication No. 2005-68132), a food containing fucosterol-3 keto form and/or fucosterol (Japanese Laid-Open Patent Publication No. 2005-104887), linseeds or their squeezed oil (Japanese Laid-Open Patent Publication No. 2004-83428), pterocarpan (Japanese Laid-Open Patent Publication No. 2003-155236), an extract from a plant in the genus Eucalyptus (Japanese Laid-Open Patent Publication No. 2001-270833), a food containing proanthocyanidin and isoflavone (Japanese Patent No. 3510526), a food containing procyanidin (Japanese Laid-Open Patent Publication No. 9-291039), and a mixture of chitosan and a peptide (Japanese Patent No. 3108675). Furthermore, examples of peptides that are being developed for the purpose of treating arteriosclerosis include a C-terminal peptide of apolipoprotein A-1 (Japanese Laid-Open Patent Publication No. 8-157492), a peptide having a serine protease inhibitory action (Japanese Laid-Open Patent Publication No. 2004-67583), and a hexapeptide (Japanese Patent No. 2803477). However, in these foods and substances having an anti-arteriosclerotic action, a cell growth suppressing effect in cultured cells, the level of hormone secreted from cells, and blood parameters such as the blood triglyceride concentration and the blood cholesterol concentration of animals, are often taken as an indicator. In other words, direct evaluation is hardly performed on the anti-arteriosclerotic action on the blood vessel structure itself taking the media/lumen ratio, or the like, as an indicator. Accordingly, the arteriosclerosis suppressing effect of these foods or substances is not necessarily sufficiently satisfactory. Moreover, these inventions include chemically synthesized substances, and thus it is feared that side effects might occur in continuous ingestion for preventing arteriosclerosis. Thus, for better safety, it is desirable to develop a naturally-occurring product-derived agent for preventing arteriosclerosis.

As a method for diagnosing arteriosclerosis in human, there is a method in which the carotid intima-media thickness (IMT) measured by ultrasonography of the carotid is used as a diagnosis criterion (Japanese Laid-Open Patent Publication No. 2005-390). It is considered that if the IMT is more than 1.1 mm, then the occurrence of cerebrovascular disorders or ischemic heart disease increases, and thus the IMT is regularly measured in some institutions. In the case of experimental animals, the media/lumen ratio of a blood vessel may be calculated and taken as an indicator for an anti-arteriosclerotic effect (K. L. Christensen et al., Journal of Hypertension, 1989, vol. 7, pp. 83-90). The smaller the value, the larger the anti-arteriosclerotic effect.

There is a therapy in which a narrowed or clogged portion of a coronary artery is dilated using a catheter whose front end is attached with a balloon. When the balloon is inflated in the narrowed portion of the coronary artery, wall faces of the blood vessel is pressed to be expanded by the pressure, and the narrowed blood vessel is improved. However, this treatment method has the problem that a treated portion causes restenosis after three to six months. The reason for this seems to be that due to a stimulation given by dilation of the blood vessel, the blood vessel becomes gradually thickened. As a method for preventing this restenosis, various methods are examined such as permanently leaving a stent (small mesh tube made of metal) at a lesion, but an innovative means has not been found yet.

Obesity, which may cause various life style-related diseases, is considered to be induced by ingestion of excessive energy and lowering of energy consumption that are caused by various factors such as overeating, lack of exercise, metabolic disorder, and heredity. Generally, in order to improve obesity, a dietary therapy is performed that restricts ingestion of excessive energy. In ingestion of diet foods used for this dietary therapy, reduced calories often lead to insufficient proteins.

Thus, various diet foods are examined so as not to allow proteins to be insufficient. For example, as a low-calorie, high-protein, and high-nutrition diet foods, a food containing a combination of protein mixtures (mucopolysaccharide-protein, lactoprotein, powdered skim milk, soybean protein isolate, corn protein and/or solubilized collagen, albumen powder, soybean peptide), carbohydrates, lipids, anti-constipation agents, vitamins, and minerals has been proposed (Japanese Laid-Open Patent Publication No. 2001-120227). It has been found that in particular when a protein degradation product treated with an enzyme is used instead of a protein, an anti-obesity action is obtained. Known examples thereof include a diet food material containing a water-soluble peptide obtained by treating beef with an enzyme (Japanese Laid-Open Patent Publication No. 10-66542), an anti-obesity food containing a soybean protein or a soybean protein degradation product instead of casein (Japanese Laid-Open Patent Publication No. 10-71), a food containing a peptide derived from a plant (barley, wheat, potato, soybean, Chinese quince, or sake lees) and a marine-derived peptide (fish peptide), the peptides being obtained by enzymatic degradation (Japanese Laid-Open Patent Publication No. 2002-142723), and a beverage containing an enzymatic degradation product of a soybean protein (Japanese Laid-Open Patent Publication No. 2002-10764).

### DISCLOSURE OF INVENTION

The present invention is based on the fact that a substance having an excellent anti-hypertensive effect, anti-arteriosclerotic effect, anti-obesity, and the like, the substance being in no danger of a side effect even after continuous ingestion, is found from a protein degradation product of mung beans, which are one type of edible beans. It is an object of the present invention to provide this substance, as a material to be added to a functional food or health food for preventing life style-related diseases, or as such a functional food or health food itself.

The present invention provides a food for preventing life style-related diseases, comprising a mung bean protein degradation product.

In one embodiment, the life style-related diseases are hypertension, arteriosclerosis, or obesity.

The present invention also provides an anti-hypertensive agent, comprising a mung bean protein degradation product.

The present invention further provides an anti-arteriosclerotic agent and a vascular intimal hypertrophy inhibitor comprising a mung bean protein degradation product.

The present invention further provides an anti-obesity agent comprising a mung bean protein degradation product.

The present invention also provides a drug and a quasi-drug comprising the anti-hypertension agent, the anti-arteriosclerotic agent, the vascular intimal hypertrophy inhibitor, or the anti-obesity agent.

Moreover, the present invention provides a method for treating and preventing life style-related diseases, comprising the step of ingesting a food, a drug, or a quasi-drug containing a mung bean protein degradation product.

Furthermore, the present invention provides a method for using a mung bean protein degradation product for treating and preventing life style-related diseases, comprising the steps of: producing a food, a drug, or a quasi-drug containing the mung bean protein degradation product; and ingesting the food, the drug, or the quasi-drug.

In one embodiment, the life style-related diseases are hypertension, arteriosclerosis, or obesity.

In an embodiment, in the mung bean protein degradation product, a dipeptide or tripeptide content in the entire peptides is 10% or more, and more preferably 50% or more. In a further embodiment, the mung bean protein degradation product is obtained by hydrolyzing a mung bean-derived protein with a protease. Furthermore, in an embodiment, the food, the drug, or the quasi-drug contains the mung bean protein degradation product at a ratio of 0.1 wt % to 100 wt %, and more preferably 1 wt % to 100 wt %.

The present invention provides a diet food containing a mung bean protein degradation product.

In an embodiment, in the diet food, a dipeptide or tripeptide content in the entire peptides of the mung bean protein degradation product is 10% or more, and more preferably 50% or more.

In one embodiment, the mung bean protein degradation product is obtained by hydrolyzing a mung bean-derived protein with a protease.

In a certain embodiment, the diet food contains the mung bean protein degradation product at a ratio of 0.1 wt % to 100 wt %, and more preferably 1 wt % to 100 wt %.

### BRIEF DESCRIPTION OF DRAWINGS

FIG. 1 shows graphs illustrating changes over time in the systolic blood pressure (A), the diastolic blood pressure (B), and the average blood pressure (C) of SHRs in a long-term administration test of a diet containing a mung bean protein degradation product.

FIG. 2 shows graphs illustrating changes over time in the systolic blood pressure (A), the diastolic blood pressure (B), and the average blood pressure (C) of SHRs during a drug termination period after a long-term administration of the diet containing the mung bean protein degradation product.

FIG. 3 shows graphs illustrating changes over time in the systolic blood pressure (A), the diastolic blood pressure (B), and the average blood pressure (C) of SHRs in a single administration test of the diet containing the mung bean protein degradation product.

FIG. 4 is a graph illustrating the amount of body weight gain of mice in each group between pre-administration and post-administration.

FIG. 5 is a graph illustrating the weight of adipose tissues around epididymis of mice in each group after administration.

FIG. 6 is a graph illustrating the weight of adipose tissues around epididymis with respect to the body weight of mice in each group after administration.

### BEST MODE FOR CARRYING OUT THE INVENTION

Mung beans, which are a raw material plant of a mung bean protein degradation product used in the present invention, are a plant belonging to the family Leguminosae. The bean portion is green to brown, and is smaller than azuki bean. Sprouts of the mung beans are known as bean sprouts, and starch of the mung beans is known as a raw material of bean-starch vermicelli (Harusame).

A raw material containing a mung bean-derived protein used in the present invention (hereinafter, referred to as a “raw material”) may be a crushed product of mung beans, a juice thereof, or an extract thereof obtained with water, an acid, or an alkali. Moreover, by-products obtained during processing of the mung beans also can be used as the raw material. Examples thereof include a by-product containing a mung bean-derived protein that is mainly composed of starch of the mung beans and produced during a production process of bean-starch vermicelli. The raw material may be in any form, such as liquid, powder, or paste. Furthermore, in addition to the mung bean-derived protein, the raw material may contain sugars, dietary fibers, salts, water, or oils and fats, for example. There is no specific limitation on the amount of the protein contained in the raw material (hereinafter, referred to as a “crude protein content”), but it is preferably 15 wt % or more, and more preferably 30 wt % or more.

Herein, the crude protein content is calculated by multiplying the amount of nitrogen in the protein by a conversion factor. The amount of nitrogen is measured using semimicro-Kjeldahl method, for example. More specifically, the crude protein content is obtained in the following manner. First, 50 mg of sample is accurately weighed in a Kjeldahl flask. Then, 10 g of potassium sulfate and 1 g of copper sulfate are mixed for forming a degradation promoter. Next, 1 g of the degradation promoter is placed in the flask, and then 5 mL of concentrated sulfuric acid is added thereto. After being allowed to stand overnight, the flask is gradually heated until the liquid is transparent and carbide is no longer observed on the inner wall of the flask. After cooling down, 20 mL of distilled water is added thereto and sufficiently mixed, and then the resultant is cooled with ice. The flask is connected to a distillation apparatus that has been washed in advance by passing water vapor. Then, 10 mL of 0.1N sulfuric acid, and two to three droplets of an indicator (mixed liquid of methyl red and methylene blue reagents) are placed in a receiving unit for receiving a distilled liquid, and the lower end of a cooler of the distillation apparatus is soaked therewith. From a funnel connected to the distillation apparatus, 20 mL of 40% sodium hydroxide is added to the flask, and distillation is performed for six to seven minutes with passing water vapor. The lower end of the cooler is removed from the liquid face, and this portion is washed with a small amount of water. Titration is performed with 0.1N sodium hydroxide. Furthermore, a blank is obtained by the same measurement method, without adding the sample. The crude protein content is calculated by the following formula:

Crude protein content (% (w/w))={([B]−[A])×F×1.4007×6.25/[C]}×100

where [A] represents the volume (mL) of 0.1N sodium hydroxide that is required for the titration when the sample is added, and [B] represents the volume (mL) of 0.1N sodium hydroxide that is required for the titration of the blank. Furthermore, [C] represents the weight (mg) of the sample, and “F” represents a factor of the sodium hydroxide used for the titration, and “1.4007” corresponds to the weight (mg) of nitrogen corresponding to 1 mL of 0.1N sulfuric acid. Furthermore, in the present invention, “6.25” was used as a protein content conversion factor from the weight of nitrogen.

In the present invention, the mung bean protein degradation product refers to a degradation product obtained by hydrolyzing a mung bean-derived protein using any means, or a degradation product obtained by degrading a mung bean-derived protein with an acid or an enzyme such as a protease. The degradation product obtained with the protease, which is a proteolytic enzyme, is preferable because the reaction is mild and by-products are produced less.

Examples of protease include: enzymes derived from bacteria, for example, the genus Rhizopus such as Rhizopus delemar and Rhizopus niveus, the genus Aspergillus such as Aspergillus niger and Aspergillus oryzae, and the genus Bacillus such as Bacillus subtilis and Bacillus sp.; animal-derived enzymes such as pepsin and pancreatin; and plant-derived enzymes such as papain and bromelain. In the present invention, acid protease derived from the genus Aspergillus is preferable because the degree of degradation is easy to control. Protease may be a commercially available purified material or a crude material, and may be used alone or in combination of two or more. Hydrolysis reaction conditions (reaction temperature, pH, time, amount of enzyme used, etc.) may be set according to the optimum action conditions of protease to be used. Usually, the temperature is 10° C. to 80° C., the pH is 2 to 11, the reaction time is 2 to 48 hours, and the amount of enzyme used is 10 to 30,000 units with respect to 1 g of the crude protein. Preferably, the reaction temperature is 30° C. to 60° C., the pH is 3 to 8, the reaction time is 4 to 20 hours, and the amount of enzyme used is 100 to 7000 units with respect to 1 g of the crude protein.

After the protease treatment, insoluble matters are removed from the obtained protease-treated product by filteration or centrifugation. The obtained supernatant is dried by methods such as vacuum concentration, freeze drying, and spray drying, and thus a mung bean protein degradation product is obtained. In the above-described process, before the drying, purification also can be performed as appropriate using activated carbon or resin, for example. The form of the obtained mung bean protein degradation product may be liquid, powder, or paste. The obtained mung bean protein degradation product usually is odorless and has a good flavor.

In the mung bean protein degradation product, the dipeptide or tripeptide content in the entire peptides is preferably 10% or more. The dipeptide or tripeptide content is more preferably 40% or more. The content is even more preferably 50% or more because the absorption is excellent when the degradation product is ingested. Herein, the dipeptides or tripeptides specifically refer to peptides whose molecular weight is in a range of approximately 130 to 580, in an analysis of the molecular weight distribution described next in detail.

In the present invention, the following methods were used for an analysis of the molecular weight distribution of the mung bean protein degradation product, and the dipeptide or tripeptide content.

The molecular weight was measured using a gel filtration column (Superdex Peptide HR 10/30, manufactured by Pharmacia Biotech), by high performance liquid chromatography. As a mobile phase, 30% acetonitrile aqueous solution containing 0.1% trifluoroacetic acid was used, the flow rate was set to 0.3 mL/min, and detection was performed based on ultraviolet region (220 nm) absorption. The molecular weight was obtained by producing a calibration curve using glycine (molecular weight 75.07) and oligopeptides each having known molecular weight, that is, alanylproline (molecular weight 186.2), angiotensin II (molecular weight 1046.2), and substance P (molecular weight 1347.7). The molecular weight distribution was indicated as the area ratio of an integration chart using a data processing apparatus (D-2500 Chromato-Integrator, manufactured by Hitachi, Ltd.). The dipeptide or tripeptide content was obtained in the following manner: the total amount of peptides whose mean molecular weight was in a range of 130 to 580 was obtained from the integration chart, this total amount was taken as the dipeptide or tripeptide amount, and then the ratio with respect to the total amount of the entire peptides was obtained.

The food for preventing life style-related diseases of the present invention contains the mung bean protein degradation product, and may be provided in the form of various foods. Herein, the life style-related diseases refer to non-infectious chronic diseases that are often taken in the middle age and thereafter. Examples thereof include: circulatory system diseases such as hypertension, arteriosclerosis, ischemic heart disease, and cerebral stoke; diabetes; and malignant neoplasms. The food for preventing life style-related diseases of the present invention is useful for preventing these life style-related diseases, and is suitable for preventing in particular hypertension and arteriosclerosis. Furthermore, the food is useful also for preventing and improving obesity, which causes these life style-related diseases. Accordingly, in the present invention, obesity also may be included in the life style-related diseases.

In addition to the mung bean protein degradation product, the food for preventing life style-related diseases of the present invention may contain proteins, fibers (including dietary fibers), starches, sugars, lipids, minerals, vitamins, and additives commonly used for foods, for example. In this case, the content of the mung bean protein degradation product in the food is preferably 0.1 to 100 wt %, and more preferably 1 to 100 wt %. Examples of such a food include: processed cereal/bean products such as porridge, bread, and thick blocks of deep-fried bean curd; processed livestock products such as sausage and ham; processed seafood products such as steamed fish paste and tubular roll of fish paste; dairy products such as yoghurt and soybean milk; processed egg products such as pudding and steamed egg hotchpotch; confectioneries such as biscuit and rice cracker; cooked and processed foods such as frozen croquette and frozen fried shrimp; beverages such as juice and cocoa powder; and seasonings such as seasonings for pot noodles, soy sauce, and sauces. These foods may be not only ordinary foods, but also health beverages and foods or foods for specified health use, for example. Moreover, these foods may be in any form such as liquid, solid, block, powder, or semiliuquid.

These foods can be sold with the indication “for those who are anxious about life style-related diseases”, “for those who have high blood pressure”, “for those who are anxious about hypertension”, “for those who have a high degree of arteriosclerosis”, “for those who are anxious about arteriosclerosis”, “for those who are slightly overweight”, “for those who are anxious about body weight”, or “for those who are anxious about body fat”, for example.

The mung bean protein degradation product can be used as an anti-hypertensive agent of the present invention. Alternatively, the mung bean protein degradation product can be used as an anti-arteriosclerotic agent and a vascular intimal hypertrophy inhibitor of the present invention. Herein, the anti-arteriosclerotic agent and the vascular intimal hypertrophy inhibitor refer to substances that can prevent, treat, or improve arteriosclerosis and vascular intimal hypertrophy, respectively. The improvement of arteriosclerosis and vascular intimal hypertrophy refers to suppression of progress of the arteriosclerosis and the vascular intimal hypertrophy. Furthermore, the mung bean protein degradation product can be used as an anti-obesity agent of the present invention.

The route of administration of a drug containing the anti-hypertensive agent, the anti-arteriosclerotic agent, the vascular intimal hypertrophy inhibitor, or the anti-obesity agent of the present invention, in which the mung bean protein degradation product serves as an active component, may be any one of oral, intrarectal, and intravenous routes. Among them, oral administration is preferable.

The anti-hypertensive agent, the anti-arteriosclerotic agent, the vascular intimal hypertrophy inhibitor, and the anti-obesity agent of the present invention may be administered such that the mung bean protein degradation product is in the original form or in the form of preparation, depending on the route of administration. The dosage form of the anti-hypertensive agent, the anti-arteriosclerotic agent, the vascular intimal hypertrophy inhibitor, and the anti-obesity agent of the present invention is selected as appropriate depending on the route of administration. Examples of the dosage form include tablets, capsules, granules, powders, syrups, suspensions, and injectable form. These preparations are prepared by a method commonly used by those skilled in the art. These preparations contain the mung bean protein degradation product at a ratio of 0.1 wt % or more, and more preferably 1 to 100 wt %.

The preparations may contain a pharmaceutically acceptable carrier used in the pharmaceutical field. Examples of the pharmaceutically acceptable carrier include excipients such as lactose, dextrin, sucrose, mannitol, corn starch, and sorbitol, and auxiliary agents such as crystalline cellulose and polyvinylpyrrolidone. These carriers may be used alone or in appropriate combination. Moreover, additives such as buffer, preservatives, anti-oxidants, flavoring agents, coloring matter, and sweetener also may be used as appropriate. The amount of these additives contained can be determined as appropriate by those skilled in the art. The preparations may contain other medicinal components that do not inhibit the anti-hypertensive action.

The dose of the anti-hypertensive agent, the anti-arteriosclerotic agent, the vascular intimal hypertrophy inhibitor, and the anti-obesity agent of the present invention varies depending on factors such as administration methods, and symptoms and age of patients. The crude protein content of the mung bean protein degradation product per day is usually 1 mg to 200000 mg, preferably 100 mg to 20000 mg, and more preferably 500 mg to 10000 mg.

The anti-hypertensive agent, the anti-arteriosclerotic agent, the vascular intimal hypertrophy inhibitor, and the anti-obesity agent of the present invention can be provided not only as a drug but also as a quasi-drug or a food.

Ingestion of the anti-hypertensive agent of the present invention and the food, drug or quasi-drug containing the anti-hypertensive agent gradually lowers the blood pressure. For example, a single administration to hypertensive rats continuously lowers the blood pressure at least for eight hours. In this manner, the effect is sustained for a relatively long time, and thus the blood pressure can be suppressed even during sleep or in the early morning during which the administration is difficult. Furthermore, in addition to the hypotensive action exhibiting a short-term effect after a single administration, an action of suppressing blood pressure elevation is provided by a long-term administration to hypertensive rats. This effect is sustained for a certain period even after termination of the administration. Thus, long-term ingestion of the anti-hypertensive agent or the food of the present invention is expected to be advantageous in that there is no danger of sudden increase of blood pressure even in the case of a failure to take these products. From these points of view, the anti-hypertensive agent of the present invention is preferable for daily and long-term ingestion.

Ingestion of the anti-arteriosclerotic agent, the vascular intimal hypertrophy inhibitor, or the food, the drug or the quasi-drug containing the anti-arteriosclerotic agent or the vascular intimal hypertrophy inhibitor of the present invention provides suppression of progress of vascular intimal hypertrophy, that is, suppression of arteriosclerosis. For example, when hypertensive rats are allowed to ingest this food for a long term, the media/lumen ratio of the thoracic aortas is kept significantly low, and the weights of blood vessels in the thoracic aortas and the abdominal aortas are lowered, compared with those of rats in a control group.

Ingestion of the anti-obesity agent, or the food, the drug or the quasi-drug containing the anti-obesity agent of the present invention can suppress obesity caused by ingestion of high-fat diets and high-calorie foods. For example, when these are given together with a high-fat diet to mice, increase of the body weight and the fat weight can be suppressed.

The mung bean protein degradation product also can be used as a diet food of the present invention. In addition to the mung bean protein degradation product, the diet food of the present invention may contain proteins, fibers (including dietary fibers), starches, sugars, lipids, minerals, vitamins, and additives commonly used for foods, for example. In this case, the content of the mung bean protein degradation product in the food is preferably 0.1 to 100 wt %. For more effective use, the content is preferably 1 to 100 wt %. For even more effective use, the content is preferably 5 to 100 wt %.

In a similar manner to that of the above-described food for preventing life style-related diseases, the diet food of the present invention may be provided as various foods containing the mung bean protein degradation product. These diet foods can be sold with the indication “for those who are anxious about body weight”, “for those who are slightly overweight”, or “for those who are anxious about body fat”, for example. The diet food of the present invention can prevent obesity caused by excessive ingestion of fats, when ingested together with a high-fat diet.

### EXAMPLES

### Production Example 1

### Preparation of the Mung Bean Protein Degradation Product 1

A dried matter of drainage containing a mung bean protein, which was a by-product obtained in the production process of bean-starch vermicelli, was ground with a mill, and was dispersed by adding tap water such that the crude protein content was 6 wt %. This dispersion was adjusted to pH 3 with a hydrochloric acid, and then acid protease (Denapsin, manufactured by Nagase ChemteX Corporation) was added thereto in an amount of 3000 units with respect to 1 g of the crude protein, and allowed to react at 40° C. for 16 hours with stirring. After completion of the reaction, the reaction mixture was heated at 80° C. to 85° C. for 20 minutes to inactivate enzymes. The enzyme reaction mixture was centrifuged, and the supernatant was adjusted to pH 8 by adding sodium hydroxide. Then, activated carbon treatment, filteration, and membrane sterilization were performed, and the resultant was freeze-dried to give a mung bean protein degradation product. The molecular weight distribution of this degradation product was measured by the above-described procedure. In this degradation product, the dipeptide or tripeptide content in the entire peptides was 59.3%.

### Production Example 2

### Preparation of the Mung Bean Protein Degradation Product 2

A dispersion having a crude protein content of 6 wt % was prepared as in Production Example 1. After this dispersion was adjusted to pH 7 with sodium hydroxide, alkaline protease (Bioprase SP15-FG (trade name), manufactured by Nagase ChemteX Corporation) was added thereto in an amount of ten thousand units with respect to 1 g of the crude protein, and the reaction mixture was reacted at 40° C. for 16 hours with gently stirring. After completion of the reaction, the reaction mixture was heated at 80° C. to 85° C. for 20 minutes to inactivate enzymes. The enzyme reaction mixture was centrifuged, and the supernatant was freeze-dried to give a mung bean protein degradation product. The molecular weight distribution of this degradation product was measured by the above-described procedure. In this degradation product, the dipeptide and tripeptide content in the entire peptides was 61.1%.

### Production Example 3

### Preparation of the Diet Containing the Mung Bean Protein Degradation Product

As a test diet for rats that were to be used in Example 1 below, the mung bean protein degradation product obtained in Production Example 1 serving as the crude protein was mixed with a CRF-1 powder diet (Oriental Yeast Co., Ltd.) such that the crude protein was contained at ratios of 0.25 w/w %, 1.25 w/w %, and 2.50 w/w % respectively, and the mixtures were shaped into pellets with a tableting machine.

### Production Example 4

### Preparation of the Mung Bean Protein degradation product 3

A mung bean protein degradation product was obtained from a dried matter of drainage containing the mung bean protein, as in Production Example 1. The molecular weight distribution of this degradation product was measured by the above-described procedure. In the degradation product obtained by Production Example 4, the dipeptide or tripeptide content in the entire peptides was 64.4%.

### Example 1

### Measurement of the Blood Pressure in a Long-Term Administration to Spontaneous Hypertensive Rats (SHRs)

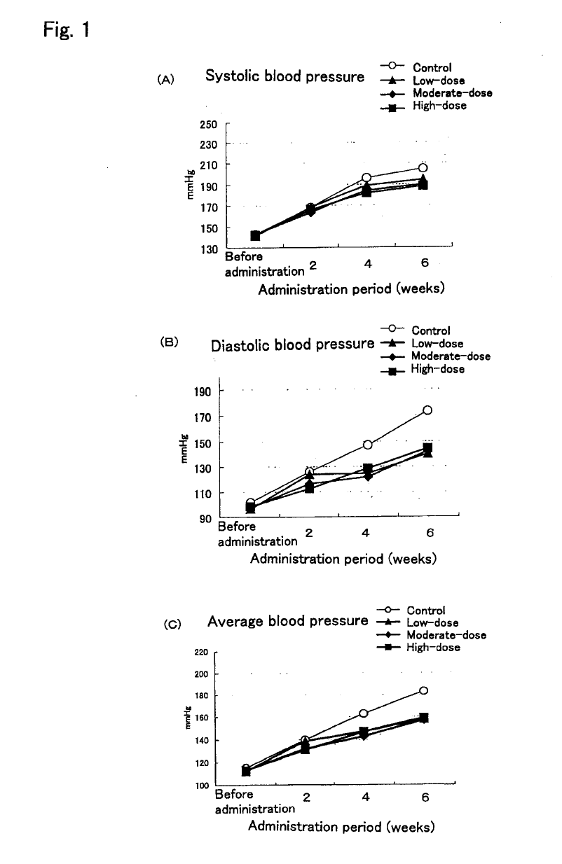

Four weeks old male spontaneous hypertensive rats (SHRs) (Japan SLC, Inc.) were allowed to freely ingest a solid diet (CRF-1: Oriental Yeast Co., Ltd.) and tap water for acclimatization for two weeks. After the acclimatization, the SHRs were divided into four groups each consisting of six rats. A low-dose group, a moderate-dose group, and a high-dose group were provided as test groups, and the other one group was taken as a control group. For six weeks, the test groups were allowed to freely ingest the diets containing the mung bean protein degradation product at the respective concentrations obtained in Production Example 3. The control group was allowed to freely ingest the CRF-1 solid diet as a normal diet. During the test period, the blood pressure, the heart rate, the food intake, and the body weight were measured at every two weeks. As the blood pressure, the blood pressures at the caudal artery and vein were measured using a noninvasive automated blood-pressure monitoring apparatus (BP-97A: manufactured by Softron). The measurement results of the diastolic blood pressure, the systolic blood pressure, and the average blood pressure are shown in Tables 1 to 3 and FIG. 1. Tables 1 to 3 show changes in the systolic blood pressure (Table 1), the diastolic blood pressure (Table 2), and the average blood pressure (Table 3) of the SHRs during the first six weeks of the long-term administration test of the diets containing the mung bean protein degradation product. The data was indicated as a mean value±standard error. As statistical analysis, analysis of variance using a Bartlett's test for equality of variance, and then a Dunnett's test were performed. The significance level was set to 5%.

TABLE 1
Systolic blood pressure (mmHg)

| Administration | | | | |
|---|---|---|---|---|
| period | Before | | | |
| (weeks) | administration | 2 | 4 | 6 |
| Control | 143 ± 2.9 | 168 ± 4.1 | 196 ± 3.4 | 205 ± 4.1 |
| Low-dose | 141 ± 3.3 | 168 ± 4.1 | 189 ± 3.3 | 194 ± 3.3 |
| group | | | | |
| Moderate-dose | 143 ± 2.7 | 163 ± 2.7 | 184 ± 3.1* | 190 ± 4.6* |
| group | | | | |
| High-dose | 142 ± 2.2 | 166 ± 4.4 | 182 ± 2.6* | 188 ± 2.4* |
| group | | | | |

*p < 0.05 (Dunnett's test)

TABLE 2
Diastolic blood pressure (mmHg)

| Administration | | | | |
|---|---|---|---|---|
| period | Before | | | |
| (weeks) | administration | 2 | 4 | 6 |
| Control | 102 ± 3.0 | 126 ± 4.2 | 147 ± 6.7 | 173 ± 7.2 |
| Low-dose | 97 ± 3.1 | 124 ± 5.7 | 125 ± 3.2* | 140 ± 4.0** |
| group | | | | |
| Moderate-dose | 98 ± 3.2 | 117 ± 2.9 | 122 ± 5.6* | 142 ± 5.2** |
| group | | | | |
| High-dose | 99 ± 2.6 | 113 ± 8.5 | 129 ± 6.5 | 144 ± 3.7** |
| group | | | | |

*p < 0.05
**p < 0.01 (Dunnett's test)

TABLE 3
Average blood pressure (mmHg)

| Administration | | | | |
|---|---|---|---|---|
| period | Before | | | |
| (weeks) | administration | 2 | 4 | 6 |
| Control | 115 ± 2.6 | 140 ± 3.7 | 163 ± 5.5 | 183 ± 6.2 |
| Low-dose | 112 ± 2.8 | 139 ± 5.9 | 147 ± 3.1 | 158 ± 3.7** |
| group | | | | |
| Moderate-dose | 113 ± 2.9 | 132 ± 2.0 | 143 ± 4.4* | 157 ± 4.9** |
| group | | | | |
| High-dose | 113 ± 2.0 | 131 ± 7.0 | 147 ± 4.9 | 159 ± 2.9** |
| group | | | | |

*p < 0.05
**p < 0.01 (Dunnett's test)

As shown in Tables 1 to 3 and FIG. 1, in the control group, the blood pressure gradually increased from the beginning of the test. On the contrary, elevation of the blood pressure was suppressed in all of the test groups. At the fourth week and thereafter, a significant effect of suppressing blood pressure elevation was observed in contrast to the control group (p<0.05). Elevation of the blood pressure was suppressed in both of the systolic blood pressure and the diastolic blood pressure, and the tendency was observed that the effect was larger in the diastolic blood pressure. From these results, it was found that the mung bean protein degradation product suppresses elevation of the blood pressure. It should be noted that no significant difference was observed between the test groups and the control group, in the heart rate, the food intake, and the body weight, during the first six weeks of the test.

### Example 2

### Change in the Blood Pressure Due to Drug Termination After a Long-Term Administration

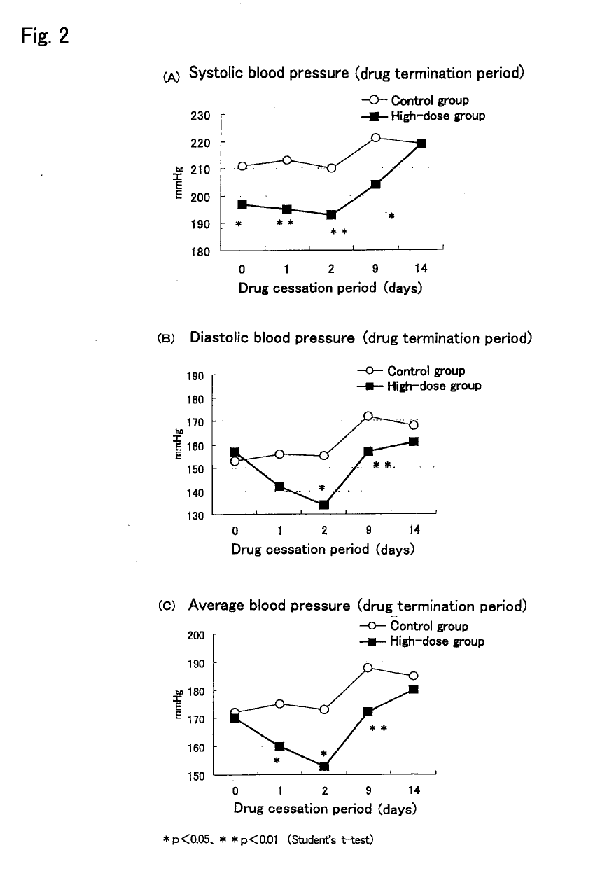

After the diets were continuously administered up to the eighth week to the control group and the high-dose group of Example 1, the diet for the high-dose group was changed into a normal diet (hereinafter, it is referred to as “drug termination”). Then, the blood pressure was measured over time for two weeks. The measurement was performed before the drug termination, one day, two days, nine days, and 14 days after starting the drug termination. The measurement results of the diastolic blood pressure, the systolic blood pressure, and the average blood pressure during the drug termination are shown in FIG. 2.

As shown in FIG. 2, it was observed that the blood pressure of the high-dose group tended to be maintained at a lower level than that of the control group during at least the first nine days of the drug termination. Accordingly, it was found that the effect of suppressing blood pressure elevation due to a long-term administration of the diet containing the mung bean protein degradation product was sustained for a while even after termination of the administration.

### Example 3

### Measurement of the Blood Pressure in a Single Administration to the Spontaneous Hypertensive Rats (SHRs)

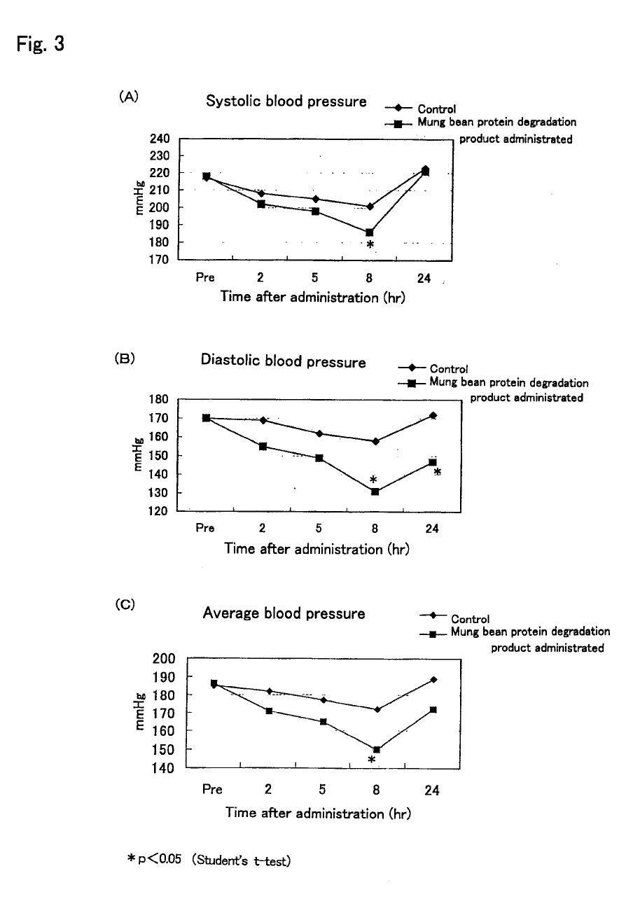

After the drug termination of Example 2, in order to completely recover the blood pressure of the high-dose group, rearing and drug termination were continued for another three weeks for the SHRs in the control group and the high-dose group of Example 1. Then, it was confirmed that there was no difference in the blood pressure between the groups, and a single administration test was performed. More specifically, the mung bean protein degradation product obtained in Production Example 1 was suspended in water for injection, and was forcibly orally administered to the SHRs (19 weeks old, male) in the high-dose group after the drug termination, using a sonde for oral administration, at 300 mg crude protein/5 mL/kg body weight. Water for injection was administered to the control group in a similar manner. The diastolic blood pressure and the systolic blood pressure of each group after the administration were measured over time. The results are shown in FIG. 3. As shown in FIG. 3, in the mung bean protein degradation product administrated group, the blood pressure was gradually lowered after the administration, and continuously lowered for eight hours after the administration. A significant difference from the control group was observed at the eighth hour after the administration (p<0.05). Accordingly, it was found that the mung bean protein degradation product exhibits a sustainable hypotensive effect. The blood pressure was lowered in both of the systolic blood pressure and the diastolic blood pressure. The tendency was observed that the effect was large in particular in the diastolic blood pressure.

### Example 4

### Continuous Administration to the Spontaneous Hypertensive Rats (SHRs)

The test diets were continuously administered to the SHRs in the low-dose group and the moderate-dose group used in Example 1. The mung bean protein degradation product was ingested over 16 weeks in total. Furthermore, the SHRs used in Example 3 were continuously reared for another four weeks in such a manner that administration of the test diet similar to that in Example 1 was resumed for the high-dose group, and the normal diet was continuously administered to the control group. More specifically, the diets of Example 1 were continuously administered for 16 to 17 weeks, to the control group, and to the test diet groups at the low dose, the moderate dose, and the high dose. In order to implement Examples 2 and 3, only the high-dose group was provided with a drug termination period for five weeks from the eighth week to the 13^{th} week.

### Example 5

### Measurement of the Weight of Blood Vessels in the Thoracic Aortas and the Abdominal Aortas in a Continuous Administration to the Spontaneous Hypertensive Rats (SHRs)

After the rearing of the SHRs in each group shown in Example 4, the rats were dissected under pentobarbital anesthesia to remove thoracic aortas and abdominal aortas. The aortas were washed with a saline solution to sufficiently remove blood, and then removed excessive water, and weighed. The measured weight was converted into the weight per body weight of each rat (ratio with respect to 100 g of body weight), and then statistical analysis (analysis of variance using a Bartlett's test for equality of variance, and then a Dunnett's test) was performed. The results are shown in Table 4.

TABLE 4

| | Thoracic aorta | Abdominal aorta |
|---|---|---|
| Control group | 37.5 ± 3.83 | 16.3 ± 1.48 |
| Low-dose group | 37.2 ± 2.22 | 16.5 ± 1.51 |
| Moderate-dose group | 30.3 ± 2.26 | 16.5 ± 0.82 |
| High-dose group | 34.2 ± 2.21 | 10.8 ± 0.75* |

Value is expressed as mg/100g body weight,
*p < 0.05

In the mung bean protein degradation product administrated groups, it was observed that the weights of the thoracic aortas and the abdominal aortas per body weight of the rats tended to be smaller than those of the control group.

### Example 6

### Measurement of the Media/Lumen Ratio of the Thoracic Aortas in a Continuous Administration to the Spontaneous Hypertensive Rats (SHRs)

Among the thoracic aortas removed in Example 5, the thoracic aortas of the moderate-dose group and the control group were subjected to hematoxylin-eosin staining. Images of stained aortas were scanned into a computer, and the media/lumen ratio was analyzed using an image analysis system (WinROOF Ver. 3.6: MITANI CORPORATION). The ratios of the test group and the control group were subjected to statistical analysis (analysis of variance using an F-test, and then a t-test). The results are shown in Table 5.

TABLE 5

| | Media/lumen ratio |
|---|---|
| Control group | 0.288 ± 0.015 |
| Mung bean protein | 0.258 ± 0.023* |
| degradation product group | |

*p < 0.05

In the test group continuously ingesting the mung bean protein degradation product, the media/lumen ratio was significantly low (p<0.05).

The above-described results clearly show that the mung bean protein degradation product suppresses arteriosclerosis of the thoracic aortas and the abdominal aortas. Thus, this degradation product is expected to prevent vascular diseases caused by vascular intimal hypertrophy and arteriosclerosis.

### Example 7

### Transition of the Body Weight of Mice Ingesting a High-Fat Containing Diet

Five weeks old male C57BL/6J mice (CLEA Japan, Inc.) were given a diet for ordinary breeding (“Rodent Diet CE-2”, CLEA Japan, Inc.) for acclimatization for two weeks. Then, the mice were divided into groups each consisting of eight mice. The diet was substituted by a test diet, and free ingestion was allowed for five weeks. In the test diet, either one of milk casein and the mung bean protein degradation product serving as the crude protein was blended with a high-fat containing basic diet (composition in Table 6: manufactured by Oriental BioService, Inc. on request) such that the crude protein was contained at a ratio of 10% (weight ratio) with respect to the total amount of the diet. The mung bean protein degradation product that was used was obtained in Production Example 4, and contained a large amount of NaCl in the production process. In order to avoid the influence of the NaCl to the test, NaCl was added to the casein diet such that the test diets had the same amount of NaCl (diet composition was listed in Table 7). For the sake of comparison, a normal diet group was provided in which a high-fat diet was not given and a normal diet was ingested.

TABLE 6
High-fat containing basic diet

| | Ratio |
|---|---|
| Material | (part by weight) |
| Corn starch | 25.08 |
| Casein | 10.66 |
| Sucrose | 27.52 |
| Cellurose powder | 6.27 |
| AIN93G mineral Mix | 4.39 |
| AIN93 vitamin Mix | 1.25 |
| L-cystine | 0.38 |
| Choline bitartrate | 0.31 |
| t-Butylquinone | 0.0018 |
| Beef fat | 22.57 |
| Cholesterol | 1.25 |
| Sodium cholate | 0.31 |

TABLE 7

| | Mixed diet | |
|---|---|---|
| | | Mung bean protein |
| Composition of diet | Case in diet | degradation product diet |
| High-fat containing basic diet | 79.75 | 79.75 |
| Milk casein (crude protein) | 10.00 | — |
| Mung bean protein degradation | — | 10.00 |
| product (crude protein) | | |
| NaCl | 1.76 | 1.76 |
| Other ingredient derived from | 2.59 | 2.42 |
| protein material | | |
| Balance (sucrose) | 5.90 | 6.07 |

Value is expressed as part by weight.

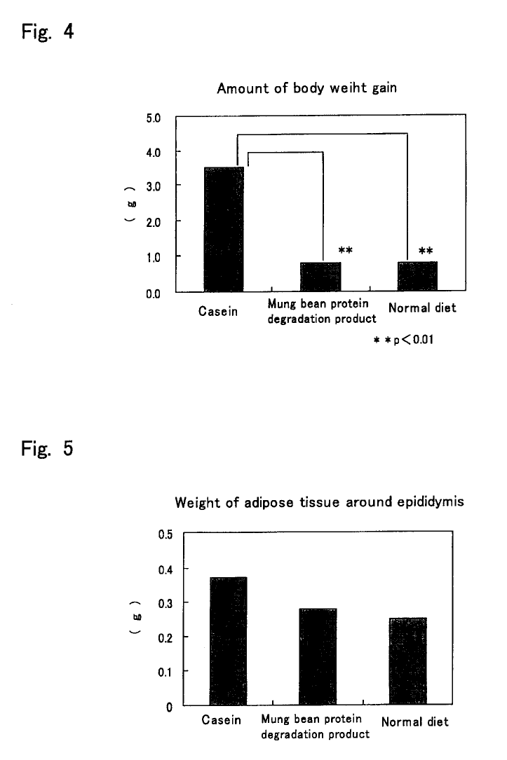

After the final administration, the mice were food-deprived for 16 hours. The body weight was measured, and then the mice were sacrificed by exsanguination under pentobarbital anesthesia. Adipose tissues around the epididymis were removed, and weighed. The measurement results of the body weight are shown in Table 8. FIGS. 4 to 6 respectively show the amount of body weight gain, the weight of adipose tissues around the epididymis, and the weight of adipose tissues around the epididymis with respect to the body weight. As statistical processing, a Kruskal Wallis rank-test was performed. If a significant difference was observed, then comparison between the groups was performed using Mann-Whitney test with Bonferroni correction. The significance level was set to 5%.

TABLE 8

| | Body weight at | Body weight after |
|---|---|---|
| | 0 day of | the end of |
| Administration group | administration (g) | administration (g) |
| Casein diet | 22.3 ± 1.3 | 25.8 ± 1.8 |
| Mung bean protein | 22.1 ± 1.1 | 22.9 ± 1.0* |
| degradation product diet | | |
| Normal diet | 22.5 ± 1.7 | 23.3 ± 1.8 |

*p < 0.05 vs casein diet

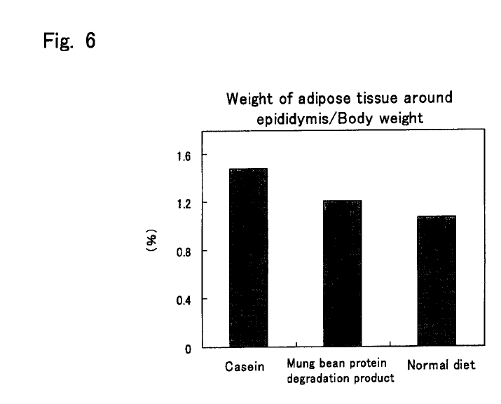

As shown in Table 8, there was no difference between the groups in the body weight before the administration. After the end of the administration, the body weight (Table 8) and the amount of body weight gain (FIG. 4) of the group ingesting the mung bean protein degradation product were significantly lower than those of the casein diet group and substantially the same as those of the normal diet group. Furthermore, in the group ingesting the mung bean protein degradation product, the tendency was observed that both of the weight of adipose tissues around the epididymis and the weight adipose tissues per body weight were lower than those of the casein diet group and substantially the same as those of the normal diet group (FIGS. 5 and 6). It should be noted that there was no significant difference in the food intake between the groups. Accordingly, it is found that the mung bean protein degradation product exhibits a superior diet effect to that of casein, which is a common animal protein.

### INDUSTRIAL APPLICABILITY

According to the present invention, a food for preventing life style-related diseases such as hypertension and arteriosclerosis is provided, the food being safe, inexpensive, and having an excellent flavor. Furthermore, according to the present invention, a food having a superior diet effect is also provided. When ingested together with a high-fat diet, the foods of the present invention can prevent obesity caused by excessive ingestion of fats, and thus can prevent life style-related diseases caused by obesity.

The anti-hypertensive agent of the present invention can be used for the purpose of suppressing elevation of the blood pressure or lowering the blood pressure, as a drug or a quasi-drug, or as foods such as health beverage and food and food for specified health use. The anti-hypertensive agent of the present invention has a sustainable hypotensive effect and an effect of suppressing blood pressure elevation. After long-term ingestion, these effects are sustained for a while even after the drug termination. Thus, the blood pressure can be suppressed even during sleep or in the early morning. Furthermore, the blood pressure does not suddenly increase even in the case of a failure to take this agent, and thus the agent is preferable for long-term ingestion.

According to the present invention, an anti-arteriosclerotic agent having an excellent anti-arteriosclerotic action and a vascular intimal hypertrophy inhibitor are provided. Moreover, an anti-obesity agent is also provided that has an action to suppress increase of the body weight and the fat weight. In a similar manner to that of the anti-hypertensive agent, these agents can be used as: a material that is to be added to a functional food or a health food; a functional food or a health food itself; a drug or a quasi-drug; or food materials of health beverages and foods or foods for specified health use, for example.

The mung bean protein degradation product used in the anti-hypertensive agent, the anti-arteriosclerotic agent, the vascular intimal hypertrophy inhibitor, the anti-obesity agent, the food for preventing life style-related diseases, and the diet food of the present invention, is obtained from naturally-occurring materials that have been conventionally used as foods. Thus, this degradation product has little side effects and a high degree of safety. Furthermore, this degradation product can be produced from a by-product obtained in the production process of bean-starch vermicelli, and thus it is environmentally friendly in view of utilizing waste materials, and is advantageous in the cost. Furthermore, in the production process, it is not necessary to perform purification using a resin, or the like, that is complicated and takes cost. Thus, also from this point of view, an advantage in the cost can be achieved.

1. A food for preventing life style-related diseases, comprising a mung bean protein degradation product.

2. The food of claim 1, wherein the life style-related diseases are hypertension, arteriosclerosis, or obesity.

3. The food of claim 1, wherein the mung bean protein degradation product is obtained by hydrolyzing a mung bean-derived protein with a protease.

4. The food of claim 1, wherein in the mung bean protein degradation product, a dipeptide or tripeptide content in the entire peptides is 10% or more.

5. An anti-hypertensive agent, comprising a mung bean protein degradation product.

6. The anti-hypertensive agent of claim 5, wherein the mung bean protein degradation product is obtained by hydrolyzing a mung bean-derived protein with a protease.

7. The anti-hypertensive agent of claim 5 wherein in the mung bean protein degradation product, a dipeptide or tripeptide content in the entire peptides is 10% or more.

8. A drug and a quasi-drug comprising the anti-hypertensive agent of claim 5.

9. An anti-arteriosclerotic agent comprising a mung bean protein degradation product.

10. The anti-arteriosclerotic agent of claim 9, wherein the mung bean protein degradation product is obtained by hydrolyzing a mung bean-derived protein with a protease.

11. The anti-arteriosclerotic agent of claim 9, wherein the mung bean protein degradation product, a dipeptide or tripeptide content in the entire peptides is 10% or more.

12. A vascular intimal hypertrophy inhibitor comprising a mung bean protein degradation product.

13. The vascular intimal hypertrophy inhibitor of claim 12, wherein the mung bean protein degradation product is obtained by hydrolyzing a mung bean-derived protein with a protease.

14. The vascular intimal hypertrophy inhibitor of claim 12, wherein in the mung bean protein degradation product, a dipeptide or tripeptide content in the entire peptides is 10% or more.

15. A drug and a quasi-drug comprising the anti-arteriosclerotic agent or the vascular intimal hypertrophy inhibitor of claim 9.

16. An anti-obesity agent comprising a mung bean protein degradation product.

17. The anti-obesity agent of claim 16, wherein the mung bean protein degradation product is obtained by hydrolyzing a mung bean-derived protein with a protease.

18. The anti-obesity agent of claim 16, wherein in the mung bean protein degradation product, a dipeptide or tripeptide content in the entire peptides is 10% or more.

19. A drug and a quasi-drug comprising the anti-obesity agent of claim 16.

20. A diet food comprising a mung bean protein degradation product.

21. The food of claim 20, wherein in the mung bean protein degradation product, a dipeptide or tripeptide content in the entire peptides is 10% or more.

22. The food of claim 21, wherein in the mung bean protein degradation product, the dipeptide or tripeptide content in the entire peptides is 50% or more.

23. The food of claim 20, wherein the mung bean protein degradation product is obtained by hydrolyzing a mung bean-derived protein with a protease.

24. The food of claim 20, containing 0.1 to 100 wt % of the mung bean protein degradation product.
